(19) United States
(12) Patent Application Publication
(10) Pub. No.: US 2009/0178150 A1
(43) Pub. Date: Jul. 9, 2009

(54) Novel Method for Generating Non-Human ES Animals

(76) Inventors: Hiroshi OHTA, Hyogo (JP); Yuko Sakaide, Hyogo (JP); Kazuo Yamagata, Hyogo (JP); Teruhiko Wakayama, Hyogo (JP)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/348,563

(22) Filed: Jan. 5, 2009

(30) Foreign Application Priority Data
Jan. 4, 2008 (JP) 2008-000089

(51) Int. Cl. C12N 15/87; A01K 67/027

(52) U.S. Cl. 800/13; 800/21

(57) ABSTRACT

The present invention provides a method for generating non-human animals by transferring ES cells to three or four tetraploid embryos to produce chimeric embryos and implanting the chimeric embryos to a psudopregnant non-human animal.

### CROSS-REFERENCE TO RELATED APPLICATION

The present application claims the benefit of priority of Japanese Patent Application No. 2008-000089 filed on Jan. 4, 2008, the entire of contents of which are incorporated herein by reference.

### BACKGROUND OF THE INVENTION

1. Field of the Invention

The present invention relates to a method for generating non-human ES animal by using tetraploid embryos as hosts and animals generated by using the method.

2. Description of Related Art

Analysis of gene function by generating genetically modified animals has been contributed to the art of basic biology and medicine. To generate genetically modified animals, it is necessary to modify the target gene mainly in ES cells and make the ES cells differentiated into germ cells. The ES cells are generally differentiated into germ cells by producing chimeric animals. In other words, this is a method by transferring ES cells (donors) to early embryos (hosts) to produce chimeric animals and then inducing differentiation to donor-derived germ cells in the chimeric animal (Capecchi MR. The new mouse genetics: altering the genome by gene targeting. Trends Genet 1989; 5:70-76). However, at the same time, this procedure results in many host-derived germ cells. Therefore, this method is needed for many crossing to obtain ES cell-derived offspring. This is a big problem to be solved.

In order to overcome this problem, a method of using tetraploid embryo-derived cells as hosts has been developed. It has been known that tetraploid embryo-derived cells can form the placenta, while can not differentiate into cells consisting of body. When chimeric embryos of ES cells and tetraploid embryos are produced by using this character, the newborn offspring is composed of ES cell-derived cells (hereinafter referred to as “ES animal”). Meanwhile, the placenta of the offspring is composed of tetraploid embryo-derived cells. Since most of cells in ES animals are derived from ES cells, their germ cells become mostly derived from ES cells (Nagy A, Gocza E, Diaz E M et al. Embryonic stem cells alone are able to support fetal development in the mouse. Development 1990; 110:815-821 and Nagy A, Rossant J, Nagy R et al. Derivation of completely cell culture-derived mice from early-passage embryonic stem cells. Proc Natl Acad Sci USA 1993; 90:8424-8428). This method allows efficiently obtaining ES cell-derived offspring. However, a problem of this method is that an efficiency of generating ES animals is quite low. As described herein, the term “ES animal” means an animal whose body is substantially composed of ES cell-derived cells which are generated by using chimeric embryos produced from ES cells and tetraploid embryos.

Approaches to enhance the generation efficiency of ES animals have been carried out mainly from a view of establishing ES cell lines. For example, it has been reported that establishing F1-derived ES cells allowed improved generation efficiency of ES animals (Eggan K, Akutsu H, Loring J et al. Hybrid vigor, fetal overgrowth, and viability of mice derived by nuclear cloning and tetraploid embryo complementation. Proc Natl Acad Sci USA 2001; 98:6209-6214). However, this method can not be used for previously established ES cells. So, there has been no effective method by using many previously-established ES cells.

### SUMMARY OF THE INVENTION

Therefore, the problems to be solved by the present invention are to provide a method for efficiently obtaining non-human ES animals and allowing use for previously-established ES cells, in case of generating non-human animals by using tetraploid embryos as hosts.

The present inventors intensively studied in order to solve the problems as described above. Surprisingly, and found that by transferring ES cells to three or four tetraploid embryos, the tetraploid embryos obtained the greatly improved function as hosts. In addition, they showed that this step could be used for previously-established ES cells.

The present invention provides:

[1] A method for generating non-human animals by following steps;

(a) transferring ES cells to three or four tetraploid embryos to produce chimeric embryos; and

(b) implanting the chimeric embryos to a pseudopregnant non-human animal.

[2] The method according to [1], wherein the number of the tetraploid embryos is three.

[3] The method according to [1], wherein a developmental stage of the tetraploid embryos is two-cell stage or four-cell stage.

[4] The method according to [1], wherein the ES cells are derived from inbred strains.

[5] A non-human ES animal, obtainable by the method according to any one of [1]-[4].

The present invention permits quite effectively obtaining non-human ES animals (more effectively by several times than prior art). Moreover, it is possible to use ES conventionally-unavailable cells such as an inbred strains-derived ES cells.

### BRIEF DESCRIPTION OF THE DRAWINGS

FIG. 1 shows a result of the cell number analysis of doubling tetraploid embryos. A-E represent photographs of PI staining of 1× to 3× tetraploid embryos and normal embryos (1×2n). A′-D′ represent photographs of Cdx2 immunostaining of 1× to 3× tetraploid embryos and normal embryos (1×2n). A″-D″ represent merge images. A-A″ show 1× tetraploid embryos, B-B″ show 2× tetraploid embryos, C-C″ show 3× tetraploid embryos and D-D″ show normal embryos (1×2n). E-E″ represent 3× tetraploid embryos stained by PI (E) and Oct3/4 (E′) and merge image (E″). Scale bars: A″-D″ 100 μm, E″ 200 μm.

FIG. 2 shows productions of chimeric embryos by using 129B6F1G1 with tetraploid embryos. A-C represent chimeric embryos of 1× tetraploid embryos and ES cells (129B6F1G1), D-F represent chimeric embryos of 3× tetraploid embryos and ES cells (129B6F1G1). A, D: optical photographs. B, E: fluorescence photographs. C, F: merge images. Scale bar: 200 μm.

FIG. 3 shows analysis of ES mice generated by using 3× tetraploid embryos. A-C represent ES mice generated from 3× tetraploid embryos expressing GFP and ES cells of E14. A: optical photograph. B: fluorescence photograph. C: merge image of A and B. Tetraploid embryo-derived (GFP-positive) cells are seen in only the placenta. D and E show results of flow-cytometry analysis. In the analysis, existence frequencies of GFP-positive cells in the brain (left panel) and liver (right panel) of 1× tetraploid embryo-derived ES mice (D) and 3× tetraploid embryo-derived ES mice (E) were determined. The existence frequencies of GFP-positive cells of both 1× and 3× tetraploid embryo-derived ES mice were less than 1%.

### DETAILED DESCRIPTION OF THE INVENTION

The present invention relates to a method for generating non-human ES animals. The animals which can be used for the present invention include all animals excluding human and preferably mammals. Examples of animals suitable for the present invention include, but not limited to, mouse, rat, guinea pig, rabbit, bovine, sheep, goat, horse and pricrosss such as rhesus, chimpanzee. The most advanced animal in the art of generating genetically modified animals such as transgenic animal or knockout animal is mouse. And, mouse is also one of the examples of the most preferred animals which can be used for the present invention.

The following is described according to the procedure for a method of the present invention.

Firstly, tetraploid embryos as hosts are prepared. As described herein, the term “host” means an animal which is same species as an ES cell-transferred animal. The tetraploid embryos can be prepared by commonly-used methods in the art e.g. fusion of embryo. The fusion of embryo may be performed by conventional means such as electro-fusion or injection. The means for fusion of embryo which can be used for the present invention is preferably electro-fusion. Moreover, the electro-fusion may be performed by a device for electro-fusion known to a skilled person in the art. For example, the electro-fusion may be performed, such that a number of two-cell stage embryos are linearly-arranged and then both sides of the embryos are sandwiched with a parallel-double electrocode. Following it, electrofusion-success embryos can be selected and cultured to obtain the tetraploid embryos. The culture condition of the tetraploid embryos can be appropriately determined by a skilled person in the art. For example, the tetraploid embryos may be cultured in CZB medium overnight.

In the present invention, for instance, two types of embryos may be fused to prepare tetraploid embryos. One of the embryos may be prepared by in vitro fertilization (IVF) known to a skilled person in the art. For example, the embryo may be generated from sperm and egg of BDF1 by IVF. As described herein, “BDF1 mouse” is a general strain generated by crossing C57BL/6 females with DBA/2 males. IVF can be performed as followed. Eggs may be collected from ovulation induction-treated female, cultured in TYH medium, and transferred to sperm as kept at constant concentration e.g. about 1×10⁵ cells/ml. Thereafter, the fertilized embryos may be cultured to develop to two-cell stage. The culture condition can be appropriately determined by a skilled person in the art. For example, the embryos may be cultured in CZB medium for 24 hours (Chatot C L, Ziomek C A, Bavister B D et al. An improved culture medium supports development of random-bred 1-cell mouse embryos in vitro. J Reprod Fertil 1989; 86:679-688). The other embryo may be generated by crossing animals. For example, the embryo may be generated by crossing female and male of ICR mice overnight, checking the crossing on the following morning, and recovering two-cell stage embryos from the oviduct of the female after 24 hours. “ICR mouse” means a mouse derived from commonly-used strains, which is characterized by bearing a lot of offspring and being good at bringing up newborn pups. Preferably, males of ICR mice have GFP gene. For example, the ICR mice include, but are not limited to, GFP transgenic mice (ICRGFP), which have a genetic background of ICR carrying pCAG-EGFP vector. Thereby, if ES animals are generated, host tetraploid embryo-derived cells are visible by expression of GFP protein.

Further, the tetraploid embryos which can be used for the present invention may be prepared from only one type of embryo. Said one type of embryo may include, but are not limited to, two-cell stage embryos produced by using IVF or crossing as described above. If desired, fusion of embryo may be performed as above.

The tetraploid embryos are placed in an appropriate vessel such as dish. Then the ES cells are transferred to the tetraploid embryos and cultured to produce chimeric embryos. When ES animals are generated by using tetraploid embryos as hosts, one or two tetraploid embryos are generally used (Nagy A, Gocza E, Diaz E M et al., Development 1990; 110:815-821 and Nagy A, Rossant J, Nagy R et al., Proc Natl Acad Sci USA 1993; 90:8424-8428). The number of the tetraploid embryos used for the present invention is preferably three or four, and more preferably three. In the present invention, if ES animals are generated by using three tetraploid embryos as hosts, the efficiency of generating ES animals is higher by 2.5-8.5 times than that using one or two tetraploid embryos. Meanwhile, if five or more tetraploid embryos are used as hosts, the efficiency is lower than that using three or four tetraploid embryos. A developmental stage of the tetraploid embryos used for the present invention may be one-cell stage just after electro-fusion or later, and is preferably from two-cell stage to morula stage. Most preferably, the developmental stage of tetraploid embryos is two-cell stage or four-cell stage. In the present invention, before the tetraploid embryos are placed in an appropriate vessel, the zona pellucida of the tetraploid embryos may be removed by using commonly-used solution such as acid Tyrode's solution. In the present invention, if a dish is used in the step of transferring ES cells, the dish may be commonly-used dish e.g. plastic dish. In this case, it is possible to enhance the generation efficiency by making a small pit per each well of the dish and supporting the tetraploid embryo by the pit when ES cells transferred. Thus, the present invention provides a vessel having such pits as supporting the tetraploid embryos and a kit for generating ES animals comprising the vessel as an essential component.

As described herein, the term “ES cell” means a cell that is derived from inner cell mass (ICM) in blastocyst stage of fertilized egg, and can be cultured and maintained with undifferentiated state in vitro. Previously, established F1-derived ES cells have been employed for efficiently generating ES animals by using tetraploid embryos as hosts (Eggan K, Akutsu H, Loring J et al., Proc Natl Acad Sci USA 2001; 98:6209-6214). However, in the present invention, various types of ES cells known to a skilled person in the art can be used. For example, the ES cells include, but are not limited to, cell lines established from 129 strains such as E14, R1 or AB-1, or commercially available cell lines such as TT2. The ES cells used for the present invention may be derived from inbred strains or hybrid strains, and are preferably inbred strains. As described herein, “inbred strains” means strains which have been brother-sister inbred over 20 times or more. Furthermore, nuclear transfer-derived ES cells (ntES cells) such as 129B6F1G1, BDmt2 or DFC3H can be used for the present invention. The ntES cells were established in the present inventor's laboratory. As described herein, 129B6F1G1 is derived from the Sertoli cell of 129B6F1 strains expressing GFP protein, BDmt2 is ntES cell from the fibroblast of BDF1 strains, and DFC3H is inbred strains-derived ntES cell established from the brain cell of C3H.

In addition, the ES cells which can be used for the present invention may include cells having ES cell-like properties (the property which can be used in the present invention), for example, including, but are not limited to, induced pluripotent stem (iPS) cells.

In the ES cells of the present invention, the target genes may be introduced or knock out.

Following the ES cells are cultured under an appropriate condition, they may be transferred to the tetraploid embryos. The ES cells may be cultured according to conventional means in the art. For instance, KOCKOUT™ DMEM (Invitrogen, CA, USA) may be used as a basal medium. In addition, 20% fetal bovine serum (Sigma-Aldrich, MO, USA), Leukemia inhibitory factor (LIF) (1000 unit/ml, Invitrogen), 1% penicillin-streptomycin (Invitrogen), 1% L-glutamine (Specialty Media, NJ, USA), 1% nonessential amino acids (Specialty Media), 1% nucleosides (Specialty Media), or 1% β-mercaptoethanol (Specialty Media) may be added to the basal medium as additional agents.

In the present invention, the ES cells may be cultured on gelatin-coated dish (in the absence of the feeder cells).

In the present invention, the number of ES cells transferred to the tetraploid embryos may be one or more. Preferably, the number of the ES cells transferred to the tetrapoid embryos is 8-15. The ES cells may be transferred as cell aggregation. The step of transferring of the present invention may be performed e.g. by contacting or infusing the ES cells with the tetraploid embryos. The above procedures are generally manipulated under the microscope or by the micromanipulator.

As described herein, “chimeric embryo” means an embryo obtained by fusion of cells derived from two or more different type of embryos. The chimeric embryos which can be used for the present invention are preferably the embryos which are obtained by transferring the ES cells to preferably three or four tetraploid embryos, followed by cultivation. The chimeric embryos may be cultured according to an appropriate condition determined by a skilled person in the art, for example in the CZB medium for about 24 hours.

In the present invention, the chimeric embryos as prepared above are implanted to pseudopregnant non-human animals to obtain ES animals. The chimeric embryos may be implanted to the ovarian duct or the uterus of pseudopregnant non-human animals. The pseudopregnant non-human animals may be same species as ES cell-derived animals. A strain of the pseudopregnant animals may be same or not as that of ES cells. For example, the pseudopregnant animals may include, but not limited to, ICR mice at 2.5 days after pseudopregnance. The chimeric embryos can be implanted to the psudopregnant animals by commonly-used means in the art. Then, according to the general procedure, the foster mothers (chimeric embryo-implanted pseudopregnant non-human animals) can be fed and bred to obtain newborn ES mice.

As described above, when three or four, particularly three tetraploid embryos as hosts are used to generate ES animals, it is possible to enhance the generation efficiency compared to the conventional means. The present invention has an advantage to be able to employ various types of ES cells e.g. inbred strains-derived ES cells. In addition, a frequency of malformed ES animals generated by using a method of the present invention is similar level to that of a conventional method. Therefore, the present method can be practiced efficiently.

The following describes materials and methods used for the example as illustrated below, but are not to be construed to limit the scope thereof.

Mice and ES Cells Used for the Present Invention

BDF1 and ICR mice purchased from SLC (Hamamatsu, Japan) were used. GFP transgenic mice having ICR background, which had been previously established by using pCAG-EGFP as vector in the present inventor's laboratory, were used. The following four types of ES cells were used. E14 (Hooper M, Hardy K, Handyside A et al., Nature 1987; 326:292-295) as commonly-used ES cells, and 129B6F1G1, BDmt2 and DFC3H as nuclear-transfer embryo-derived ES cells (ntES cell) were used. E14, which has been established by Dr. Martin Hooper (Edinburgh, Scotland) in 1987 and cultured and stocked by Dr. Peter Mombaerts (Rockefeller University), is a generous gift of Dr. Peter Mombaerts. 129B6F1G1 is derived from the Sertoli cell of 129B6F1 strains expressing GFP and BDmt2 is derived from the fibroblast of BDF1 strains, respectively. 129B6F1G1 and BDmt2, which had been previously established in the present inventor's laboratory, were used. In addition, as inbred strains-derived ntES cells, randomly selected 10 strains of ntES cell (DFC3H), which had been established from brain cells of C3H, were used to generate ES mice.

Culture Conditions of the ES Cells

The ES cells were cultured according to commonly-used condition in the art. KOCKOUT™ DMEM (Invitrogen, CA, USA) was used as base medium. In addition, the following addictive agents were added to use as the culture medium; 20% fetal bovine serum (Sigma-Aldrich, MO, USA), Leukemia inhibitory factor (LIF) (1000 unit/ml, Invitrogen), 1% penicillin-streptomycin (Invitrogen), 1% L-glutamine (Specialty Media, NJ, USA), 1% nonessential amino acids (Specialty Media), 1% nucleosides (Specialty Media), and 1% P-mercaptoethanol (Specialty Media). The ES cells were cultured on the gelatin-coated dish in the absence of feeder cells.

Production of the Tetraploid Embryos and Generation of ES Mice

The tetraploid embryos were produced from two types of the embryos in this study. One embryo (BDF2) prepared from the sperm and egg of BDF1 by in vitro fertilization (IVF) and the other embryo crossed ICRGFP male with ICR female were used. Since the latter embryo (ICRGFP) has GFP expression, the tetraploid embryo-derived cells are visible as green fluorescence. IVF was performed by commonly-used procedure. Specifically, the sperms were recovered from the cauda epididymis of BDF1 mice to culture in TYH medium (Toyoda Y, Yokoyama M, Hoshi T., Jpn J Anim Reprod 1971; 16:147-151). The eggs were collected from ovulation induction-treated BDF1 female to culture in TYH medium. Here, IVF was performed, such that the sperms were added to the medium culturing the eggs to reach 1×10⁵ cells/ml. Following IVF, fertilized embryos were collected and cultured in CZB medium for 24 hours to obtain two-cell stage embryos of BDF1 (Chatot C L, Ziomek C A, Bavister B D et al., J Reprod Fertil 1989; 86:679-688). On the other hand, in order to obtain the ICRGFP embryos, male of ICRGFP mice were crossed with ovulation induction-treated female overnight. Next morning, crossing was checked. Then the ovarian duct was collected after 24 hours, and ICRGFP at two-cell stage were recovered from the ovarian duct. BDF2 and ICRGFP at two-cell stage were fused with the Electro Cell Fusion Generator (Model LF101, Nepa gene, Chiba, Japan) to product the tetraploid embryos. The electrofusion-success embryos were selected and cultured in CZB medium overnight.

The tetraploid embryos developed to 2-4 cell stage were selected and the zona pellucida of the tetraploid embryos was removed by using acid Tyrode's solution. Then, one to three (3×) tetraploid embryos were placed in small pits per well made on a plastic dish. Subsequently, 8-15 of ES cells as cell aggregation were transferred on the pits to produce the chimeric embryos of ES cells and tetraploid embryos. After 24 hours culture in CZB medium, the resulting chimeric embryos were implanted to the uterus of pseudopregnant mice (at 2.5 days after pseudopregnance). These mice were dissected with cesarian section at 18 days after copulation and determined if newborn offspring was present.

The Cell Number Analysis of Blastocyst-Stage Embryos

To count the cell number of blastocyst-stage embryos, the embryos were determined for propidium iodide (PI) staining and immunofluorescence staining against Cdx2. PI was used as an indicator for the total number of cells because all cells were stained. Anti-Cdx2 antibody was used as an indicator for the cell number of the trophectoderm (TE) because the TE cells were stained. The cell number of the inner cell mass (ICM) was calculated as a value that subtracted the cell number of TE (Cdx2-positive cells) from the total number of cells (PI-positive cells). Specifically, 1× to 3× tetraploid embryos were developed to blastcyst stage and fixed in 4% paraformaldehyde solution at room temperature for 45 minutes. After three washes at in PBS, the embryos were incubated with anti-Cdx2 antibody (1:200; BioGenex, CA, USA) at room temperature for one hour. After three washes in PBS, the embryos were incubated with the secondary antibody (goat anti-mouse IgG conjugated with Alexa Fluor 488; Molecular Probes, Eugene, Oreg., USA) at room temperature for 30 minutes. After three washes in PBS, the embryos were transferred to 1 μg/ml of PI solution and each of the blastocyst-stage embryos were observed with a confocal microscope. To count the cell number, the confocal images of the embryos were three-dimensionally reconstructed by using MetaMorph software (Universal Imaging Co., Downingtown, Pa., USA). Then, the numbers of PI-positive cells and TE-positive cells were counted. In each count, ten blastcyst-stage embryos derived from normal embryos (1×2n) and 1× to 3× tetrapoid embryos were used.

Analysis of Newborn ES Mice

The ES mice were obtained by dissecting with cesarian section at 18 days after copulation and determined for body weight, placental weight and deformities (abdominal herniation, eye-opening and large offspring) of the newborn ES mice. In the case of using tetraploid embryos of ICRGFP as hosts, presence of tetraploid embryos-derived cells was determined by using flow cytometry with the liver and brain of ES mice. Concretely described, the liver and brain were recovered from newborn ES mice, cut finely with scissor, and incubated in 0.25% tripsin-EDTA solution at 37° C. for 15 minutes. After equal amount of DMEM containing 10% FBS were added to the solution, the solution was centrifugated at 300 g for 7 minutes. After removing the supernatant, the precipitates were washed twice in PBS. The cells of the precipitations were suspended in 10% FBS-containing DMEM and subjected to flow cytometry. In the flow cytometry, main cell groups of each tissue were identified according to side scatter (SSC) and forward scatter (FSC) by using FACSaria (BD Biosciences, San Jose, Calif., USA). That is, tetraploid embryos-derived cells were identified by counting the number of GFP-positive cells in main cell groups.

### EXAMPLES

The following examples illustrate the present invention in more detail, but are not to be construed to limit the scope thereof.

### Example 1

### Comparison of Birth Rate of Doubling Diploid Embryos

The purpose of this study is to enhance the birth rate of ES mice by using multiple tetraploid embryos. For that purpose, doubling diploid embryos (3× and 5×) were generated by using normal embryos and determined for their developmental capacities. As a result, newborn mice derived from 3× normal embryos were successfully obtained with normal rate (50%; 8/16). Meanwhile, the mice derived from 5× normal embryos were not obtained (0%; 0/8). These results suggested that an excessive number of tetraploid embryos disturbed developmental capacities. Based on these findings, birth rate of ES mice was investigated by using the chimeric embryos produced from 1× to 3× tetraploid embryos and ES cells in following study.

### Example 2

### Cell Number Analysis of Doubling Tetraploid Embryos

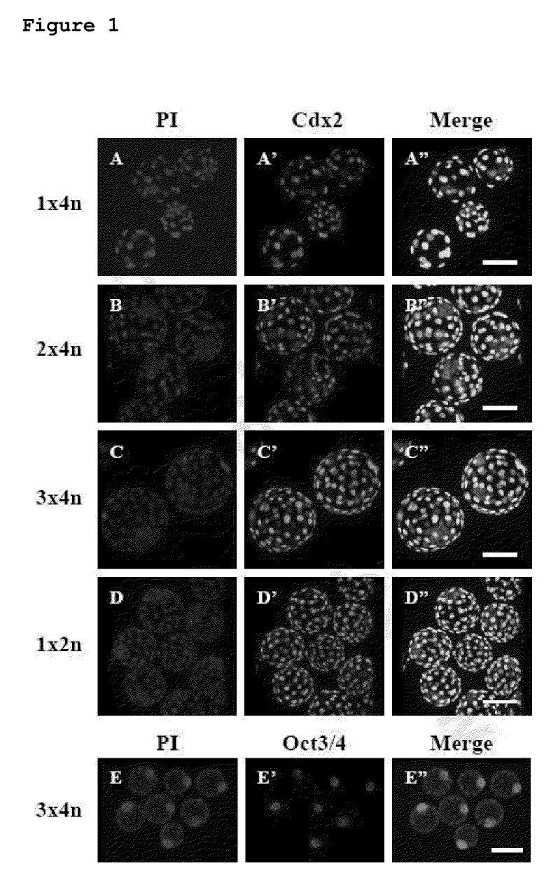

Prior to generating ES mice, it was determined if cell numbers of doubling tetraploid embryos were increased. Each ten of 1× to 3× tetraploid embryos at 96 hours after fertilization were double-stained by PI and Cdx2. Consistent with previous report (Koizumi N, Fukuta K., 1995; 44(2):105-109), it was recognized that the total number of cells of 1× tetraploid embryos was decreased compared to normal embryos (see table 1 and FIGS. 1A-A″, D-D″). Meanwhile, it was confirmed that the total numbers of cells of 2× and 3× tetraploid embryos were increased (see table 1 and FIGS. 1B-B″, C-C″). In the 2× and 3× tetraploid embryos, the cell numbers of TE and ICM were also increased, respectively (see table 1). As 3× tetraploid embryos were immunofluorescence-stained for anti-Oct3/4 antibody, ICM cells were normally stained (see FIG. 1E-E″). These results showed that the doubling tetraploid embryos in blastocyst-stage were increased with respect to cell numbers (FIG. 1) as expected and were normally differentiated into the ICM (FIG. 1E-E″).

TABLE 1
Cell number analysis of doubling tetraploid embryos

| | Total number | Cell numbers | Cell numbers |
|---|---|---|---|
| Types of | s of cells | of TE | of ICM |
| embryos | (PI staining) | (Cdx2 staining) | (Total cell − TE) |
| Normal embryos | 62.3 ± 4.8 | 58.8 ± 4.4 | 3.8 ± 2.9 |
| (1x 2n) | | | |
| 1x 4n | 23.8 ± 4.1 | 21.8 ± 4.5 | 2.2 ± 1.8 |
| 2x 4n | 58 ± 5.8 | 50.9 ± 5.5 | 7.1 ± 3.9 |
| 3x 4n | 80.5 ± 11.9 | 70.4 ± 13.1 | 10.1 ± 4.4 |

### Example 3

### Determination for Birth Rates of ES Mice by Using Doubling Tetraploid Embryos

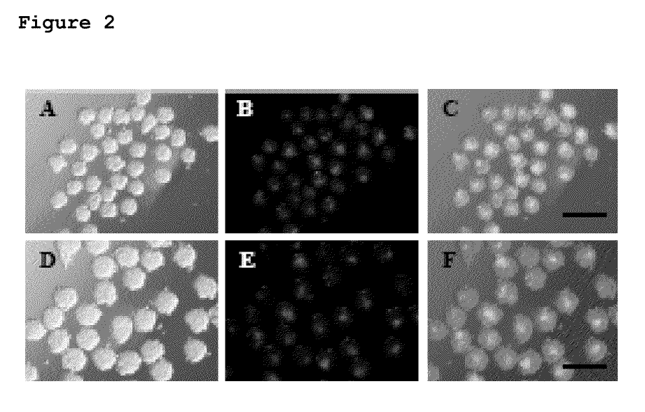

Since the above results showed that cell numbers of doubling tetraploid embryos were increased, birth rates of ES mice were determined. ES cells used for the present example were E14 commonly-used ES cell line, and 129B6F1G1 (GFP-positive) and BDmt2 as ntES cell lines, and DFC3H (total 10 lines were randomly-used) as inbred strains-derived ES cell lines. As showed in FIG. 2, when chimeric embryos were produced by using 129B6F1G1 expressing GFP, ES cells were normally introduced to 3× tetraploid embryos (see FIGS. 2D-F).

Then, chimeric embryos were produced by using the above ES cells and 1× to 3× tetraploid embryos, and determined for developmental capacity after embryo implantation. In the case of using 1× and 2× tetraploid embryos as hosts, 3 strains of ES cells (E14, 129B6F1G1 and BDmt2) showed that birth rates of ES mice were quite low (appropriately 1-3%: table 2). Meanwhile, in the case of using 3× tetraploid embryos, birth rate was increased by appropriately 2.5-8.5 times (8.6%: table 2). In addition, it was possible to generate ES mice at appropriately 7% of birth rate by using 3× tetraploid embryos though inbred strains-derived ES cells were difficult to generate ES mice (see table 2).

These results indicated that generation efficiency of ES mice by using 3× tetraploid embryos was the most highest (table 2) and 1× and 2× tetraploid embryos were insufficient (table 1) though 2× tetraploid embryos have similar cell numbers to normal embryos.

TABLE 2
Determination of the birth rate of ES mice
by using doubling tetraploid embryos

| | Types of | Numbers of | Numbers of | |
|---|---|---|---|---|
| Types of | tetraploid | transplanted | newborn | |
| ES cells* | embryos | embryos | offspring (%) | |
| E14 | 1xICRGFP | 289 | 2 | (0.7) |
| | 2xICRGFP | 102 | 4 | (3.9) |
| | 3xICRGFP | 105 | 15 | (14.3) |
| 129B6F1G1 | 1xBDF2 | 103 | 1 | (1) |
| | 2xBDF2 | 107 | 6 | (6) |
| | 3xBDF2 | 108 | 10 | (9.3) |
| BDmt2 | 1xBDF2 | 110 | 2 | (2) |
| | 2xBDF2 | 105 | 1 | (1) |
| | 3xBDF2 | 141 | 8 | (5.7) |
| DFC3H | 2xBDF2 | 51 | 1 | (2) |
| | 3xBDF2 | 120 | 8 | (6.7) |
| Total | 1x | 502 | 5 | (1) |
| | 2x | 365 | 12 | (3.3) |
| | 3x | 474 | 41 | (8.6) |

*E14; generally used ES cell. 129B6F1G1 and BDmt2; ntES cells. DFC3H; ntES cell derived from inbred strains.

### Example 4

### Analysis of Newborn Offspring Generated from Doubling Tetraploid Embryos

Since it has been known that newborn ES mice had a high frequency of deformities, phenotypes of ES mice generated by using 3× tetraploid embryos were investigated. 3× tetraploid embryos-derived ES mice had similar body weight and placental weight to 1× or 2× tetraploid embryo-derived ES mice (see table 3). In addition, there were not differences in an appearance rate of abdominal herniation, eye-opening and large offspring (see table 3). These results indicated that frequency of deformities in ES mice generated by using 3× tetraploid embryos was at least similar with that of conventional means.

The greatest characteristic of ES mice is that most of cells consisting of the body are derived from ES cells. As described in table 1, cell numbers of ICM, which would consist of the body, as well as total cell numbers were increased in the 3× tetraploid embryos. Lastly, it was determined if 3× tetraploid embryos-derived ES mice were actually composed of ES cell-derived cells.

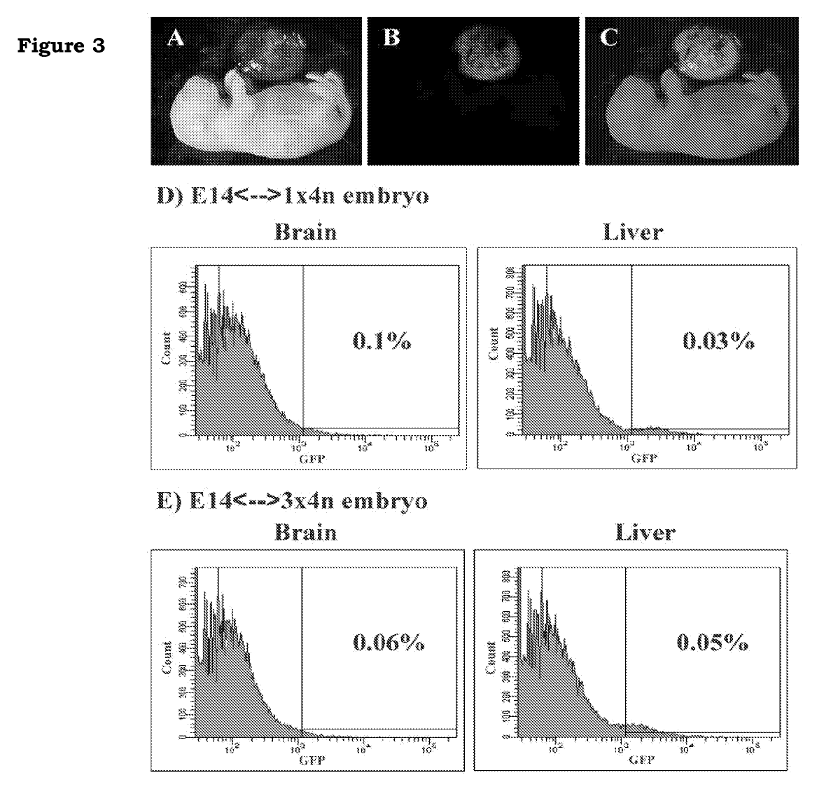

To distinguish ES cell-derived cells from tetraploid embryo-derived cells as hosts, E14 as ES cells and ICRGFP-derived tetraploid embryos as hosts were used. Therefore, the tetraploid embryo-derived cells can be readily distinguished by GFP expression. Using this procedure, total 15 of ES mice were generated from 3× tetraploid embryos and observed under the fluorescent microscope. As described in FIG. 3, GFP was expressed in the placenta, but not expressed in all of resulting 15 newborn mice (see FIGS. 3A-C). These results suggested that contamination of host tetraploid embryo-derived cells in ES mice generated by using 3× tetraploid embryos was quite rare. To investigate in more detail, GFP-positive cells were detected by using flow cytometry. Each two of the ES mice generated from 1× and 3× tetraploid embryos were used to detect GFP-positive cells in the liver and brain. In both 1× and 3× tetraploid embryo-derived ES mice, contamination rate of tetraploid embryo-derived GFP-positive cells was less than 1%.

These data showed that most of the body of 3× tetraploid embryo-derived ES mice were composed of ES cell-derived cells as well as ES mice generated by conventional means.

TABLE 3
Analysis of phenotypes of ES mice

| Types of | Body | Placental | | | | | | % of |
|---|---|---|---|---|---|---|---|---|
| host | weight | weight | Numbers of deformilities pups^{b} | | | | | abnormal |
| embryo | (n)^{a} | (n)^{a} | OE | AH | LO | OELO | OEAH | pups |
| 1x 4n | 1.75 ± | 0.33 ± | 1 | 0 | 2 | 0 | 0 | 60% |
| | 0.42 (5) | 0.08 (5) | | | | | | |
| 2x 4n | 1.65 ± | 0.26 ± | 1 | 1 | 3 | 0 | 0 | 42% |
| | 0.5 (12) | 0.08 (12) | | | | | | |
| 3x 4n | 1.66 ± | 0.26 ± | 5 | 2 | 5 | 1 | 2 | 44% |
| | 0.39 (34) | 0.06 (34) | | | | | | |

^{a}means ± SD(g).
^{b}OE: opening-eye, AH: abdominal herniation, LO: large offspring, OELO: pups having both opening-eye and large offspring, OEAH: pups having both opening-eye and abdominal herniation.

The present invention provides a method for generating genetically modified non-human animals such as transgenic animals and knockout animals, and genetically modified non-human animals generated by the method. Therefore, the present invention can be used in many industrial fields of generation of model animals for pathological study and development of new therapy and research of new medicine.

What is claimed is:

1. A method for generating non-human animals by following steps;
(a) transferring ES cells to three or four tetraploid embryos to produce chimeric embryos; and
(b) implanting the chimeric embryos to a pseudopregnant non-human animal.

2. The method according to claim 1, wherein the number of the tetraploid embryos is three.

3. The method according to claim 1, wherein a developmental stage of the tetraploid embryos is two-cell stage or four-cell stage.

4. The method according to claim 1, wherein the ES cells are derived from inbred strains.

5. A non-human ES animal, obtainable by the method according to any one of claims 1-4.
